(19) United States
(12) Patent Application Publication
(10) Pub. No.: US 2007/0010745 A1
(43) Pub. Date: Jan. 11, 2007

(54) Mechanism for ergonomic positioning of a control panel for a diagnostic ultrasound system

(75) Inventors: John Foot, San Jose, CA (US); Richard W. Henderson, Fremont, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/155,049

(22) Filed: Jun. 16, 2005

(51) Int. Cl. A61B 8/00

(52) U.S. Cl. 600/443

(57) ABSTRACT

A diagnostic ultrasound system having an ergonomic positioning mechanism is provided. The ergonomic positioning mechanism includes a table for supporting a control panel used to provide input to the ultrasound system. The table is adapted to be rotatable and translatable such that a user can position the table and control panel in an ergonomically comfortable position while performing an ultrasound.

### BACKGROUND

This invention relates to a diagnostic ultrasound systems. In particular, this invention relates to a diagnostic ultrasound system having an ergonomic positioning mechanism for allowing a control panel to be located in a comfortable position relative to a user.

Diagnostic ultrasound systems typically include a box-shaped housing for storing electrical components of the ultrasound system. The box-shaped housing generally has casters attached thereto so that the system can be easily transported to various locations when it is needed. A control panel or keyboard, used to adjust the various settings of the ultrasound system, extends from one of the four sides of the housing. A viewable display is provided on top of the housing.

Typical ultrasound systems are used at a patient's bedside such that the bedframe or the retaining bar may make it difficult to maneuver the system to a position that does not require the user to strain to move the ultrasound transducer at the same time as making adjustments via the control panel. The user needs easy access to the control panel while performing the ultrasound, but typical ultrasound systems do not provide the user with the flexibility of performing both tasks easily and simultaneously.

Ultrasound systems are typically used in a constrained environment, such as a small room with a bed and other medical equipment. As such, there is generally very little space in which to align the ultrasound system, thereby making it more difficult for the user to simultaneously perform the ultrasound and make adjustments on the control panel without undue strain.

### BRIEF SUMMARY

The present invention solves one or more of the shortcomings presented above by providing a diagnostic ultrasound system that allows the user to perform the ultrasound examination while having access to a control panel in a generally ergonomic manner. An adjustable mechanism may allow the user to move or place the control panel into a position within a confined space for easy access to the control panel while maneuvering the ultrasound transducer. The embodiments described herein also provide an adjustable mechanism that is configured to conceal cables of the ultrasound system.

In one aspect, an ergonomic positioning mechanism is provided. The ergonomic positioning mechanism includes a platform operatively connected to a component cart, wherein the electronic components of an ultrasound system are located in or on the component cart. The ergonomic positioning mechanism further includes a first arm operatively connected to the platform, a second arm operatively connected to the first arm, and a table operatively connected to the second arm. The table is moveable relative to the platform.

In another aspect, a diagnostic ultrasound system is provided. The diagnostic ultrasound system includes a component cart in or on which electronic components of the ultrasound system are located and an ergonomic positioning mechanism that is operatively connected to the component cart. The ergonomic positioning mechanism includes a table configured to support a control panel that is connected to the ultrasound system by at least one cable. The ergonomic positioning mechanism includes a stop mechanism adapted to secure the table in a substantially fixed position during transport of the diagnostic ultrasound system.

Advantages will become more apparent to those skilled in the art from the following description of embodiments which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a top perspective view of one embodiment of a diagnostic ultrasound system;

FIG. 2 is a top perspective view of an alternative embodiment of a diagnostic ultrasound system;

FIG. 3 is an exploded view of the components of one embodiment of an ergonomic positioning mechanism;

FIG. 4 is a side view of the ergonomic positioning mechanism of FIG. 3;

FIG. 5 is a perspective view of one embodiment of a platform of the ergonomic positioning mechanism;

FIG. 6 is a cross-sectional view of one embodiment of the platform of FIG. 5 shown along the line 6-6′;

FIG. 6A is a cross-sectional view of an alternative embodiment of the platform of FIG. 5 shown along the line 6-6′;

FIG. 7 is a top view of one embodiment of a first arm of the ergonomic positioning mechanism;

FIG. 7A is a bottom view of the first arm of FIG. 7;

FIG. 8 is a top view of an alternative embodiment of a first arm;

FIG. 9 is a top view of one embodiment of a second arm of the ergonomic positioning mechanism;

FIG. 9A is a bottom view of the second arm of FIG. 9;

FIG. 10 is a top view of an alternative embodiment of a second arm;

FIG. 11 is a bottom view of a table of the ergonomic positioning mechanism;

FIG. 12 is a top view of a platform having a stop mechanism attached thereto;

FIG. 13 is a side view of the stop mechanism of FIG. 12;

FIG. 14 is a magnified view of one embodiment of a connection between the stop mechanism and the table;

FIG. 15 is a top view of the ergonomic positioning mechanism in the home position;

FIG. 16 is a top view of the ergonomic positioning mechanism having the table extended in the forward direction;

FIG. 17 is a top view of the ergonomic positioning mechanism having the table extended in a lateral direction;

FIG. 18 is a top view of the ergonomic positioning mechanism having the table extended in the lateral direction opposite that shown in FIG. 17;

FIG. 19 is a top view of the rotational movement of the table when the table is translated to the position shown in FIG. 14;

FIG. 20 is a top view of the rotational movement of the table when the table is translated to the position shown in FIG. 17;

FIG. 21 is a top view of a diagnostic ultrasound system located adjacent to a patient's bed;

FIG. 22 is a top view of the platform, first arm, second arm, and stop mechanism of the ergonomic positioning mechanism in the home position.

### DETAILED DESCRIPTION OF THE DRAWINGS AND PRESENTLY PREFERRED EMBODIMENTS

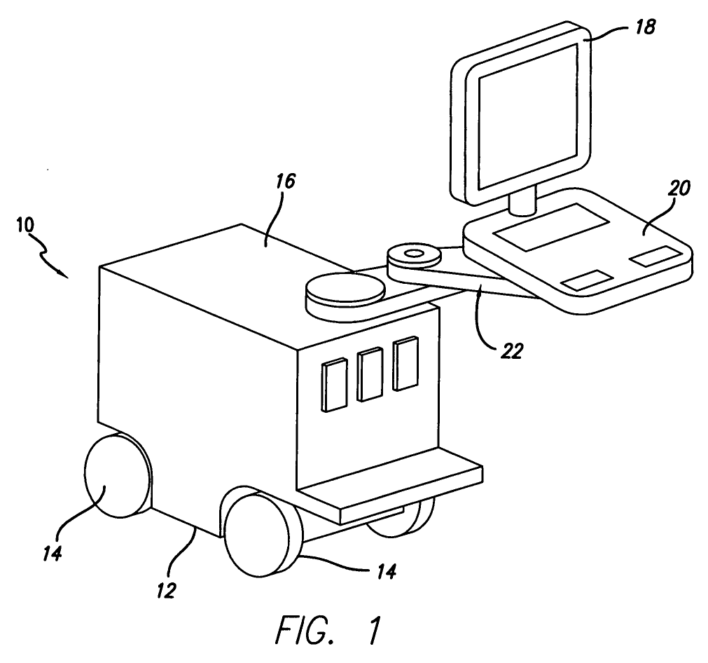

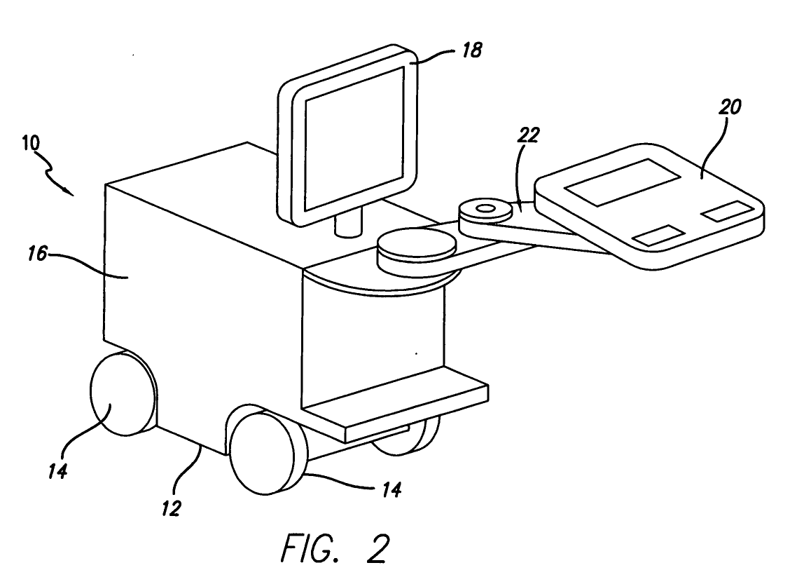

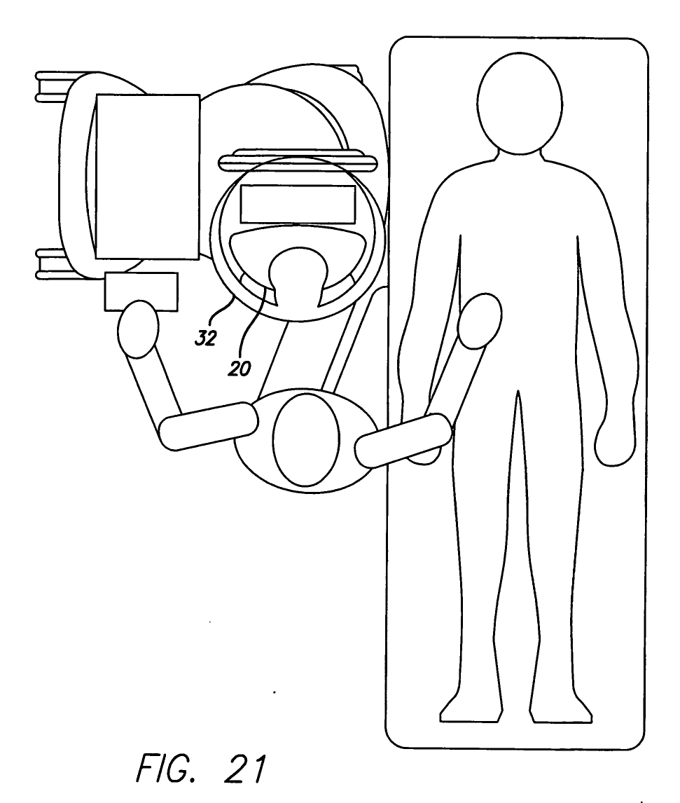

Referring to FIG. 1, one embodiment of the components of an ultrasound component cart 10 for a diagnostic ultrasound system is shown. The ultrasound component cart 10 includes a base 12, casters or wheels 14, and a housing 16. Fewer, different, or additional components may be included in the ultrasound component cart 10. The wheels 14 are connected to the base 12 to allow the ultrasound component cart 10 to be easily transportable between various locations. The base 12 supports the entire system thereon and is configured to provide a stable foundation yet be small enough to fit within crowded rooms. The housing 16 is configured to enclose the processor (not shown) for the diagnostic ultrasound system so as to protect the components of the processor. A monitor 18 is operatively connected to the housing 16, and the monitor 18 is configured to provide a user with an electronic display of the ultrasound results. A control panel 20 is connected to the processor to allow a user to provide input to the ultrasound system during, before, or after use of an ultrasound transducer. The monitor 18 and control panel 20 are connected to the processor located within the housing 16 by way of electrical wiring (not shown). In one embodiment, the monitor 18 and the control panel 20 are operatively connected to the housing 16 by way of an ergonomic positioning mechanism 22 that allows the monitor 18 and the control panel 20 to be adjusted relative to the ultrasound component cart 10, as illustrated in FIG. 1. In an alternative embodiment, the monitor 18 and the control housing 20 are connected to the housing 16 in an independent manner, as illustrated in FIG. 2. The movement of the ergonomic positioning mechanism 22 allows the user to move the control panel 20 into a comfortable position for the user (FIG. 21).

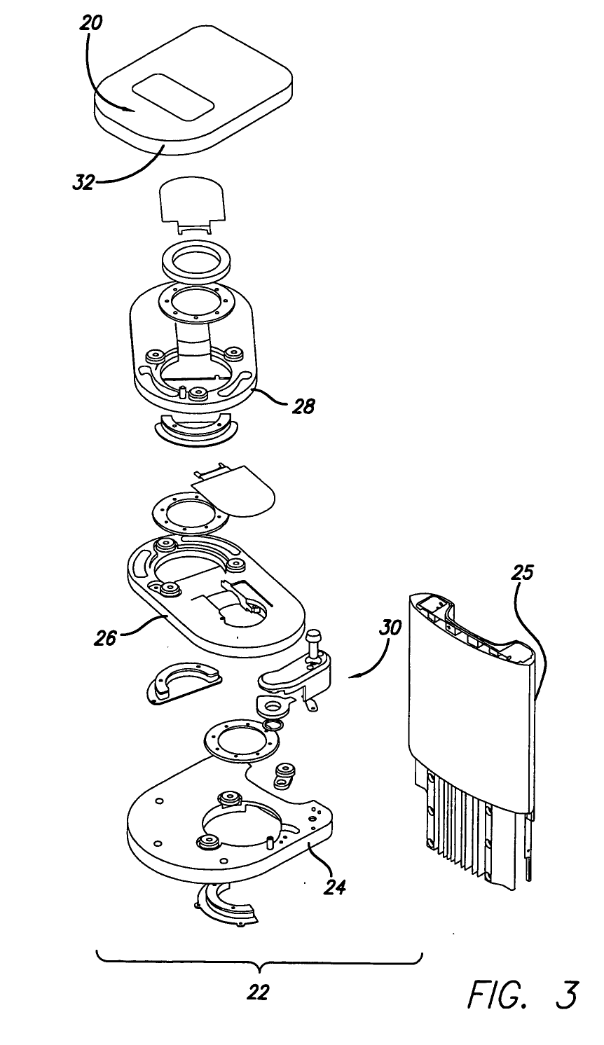

The ergonomic positioning mechanism 22, as shown in an exploded view in FIG. 3, allows the user to adjust the control panel 20 to an ergonomically efficient position such that the user can more likely access the control panel 20 while maneuvering the ultrasound transducer (not shown) across a patients body without causing undue stress on the user from stretching or contorting to perform both activities simultaneously. In one embodiment, the ergonomic positioning mechanism 22 includes a platform 24, a first arm 26, a second arm 28, a stop mechanism 30, and a table 32. Additional, different, or fewer components may be provided.

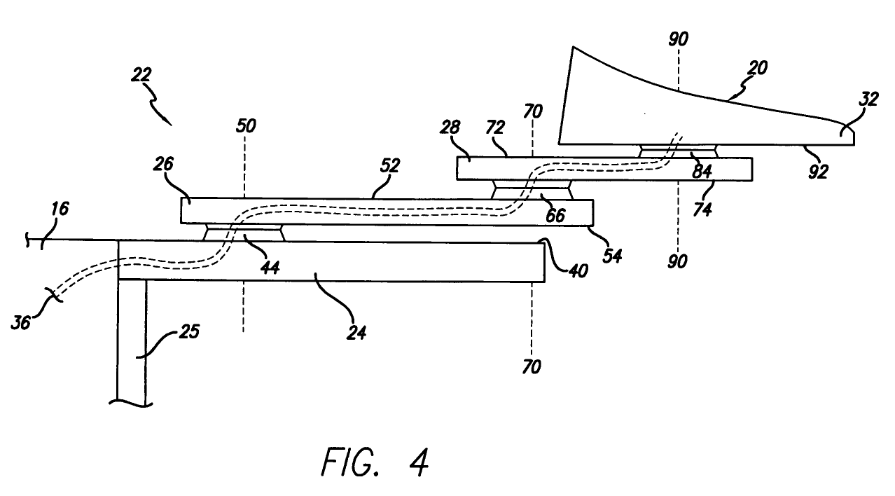

The platform 24, as illustrated in FIGS. 3-6, is operatively connected to the housing 16 and extends from the housing 16 in a cantilevered manner. In one embodiment, the platform 24 is integrally formed with the housing 16, wherein the platform 24 is an extension from the housing 16, as illustrated in FIG. 2. In an alternative embodiment, the platform 24 is attached to a lift mechanism 25 that provides vertical adjustment of the ergonomic positioning mechanism 22 relative to the housing 16, as illustrated in FIGS. 3-4. The platform 24 can be configured to translate, rotate, or be raised and lowered relative to the housing 16 so as to provide the ergonomic positioning mechanism 22 with a greater range of motion. The platform 24 is an aluminum casting, but can be made of steel, magnesium, reinforced plastic, or any other material sufficient to support the remaining components of the ergonomic positioning mechanism 22. The platform 24 can be operatively connected to the housing 16 by a variety of other devices sufficient to support and transfer the load from the ergonomic positioning mechanism 22 to the housing 16.

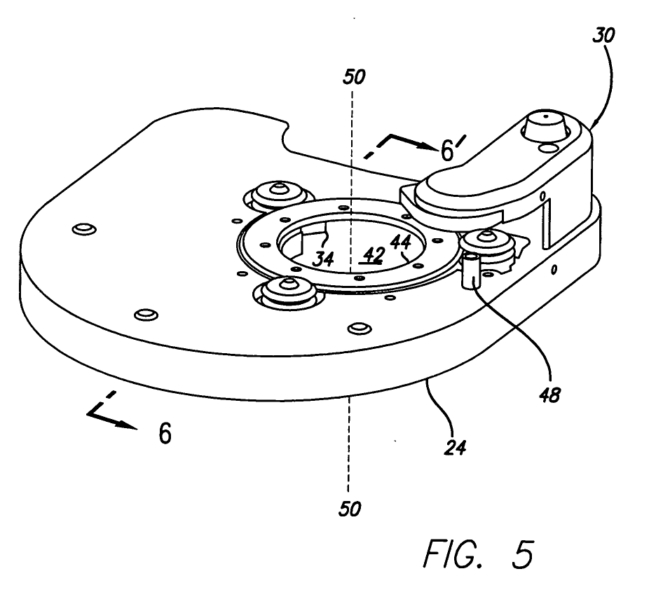

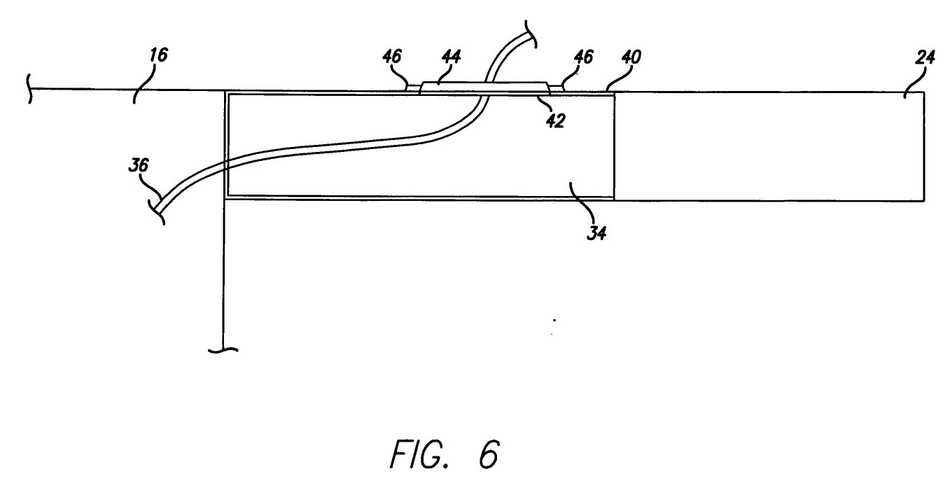

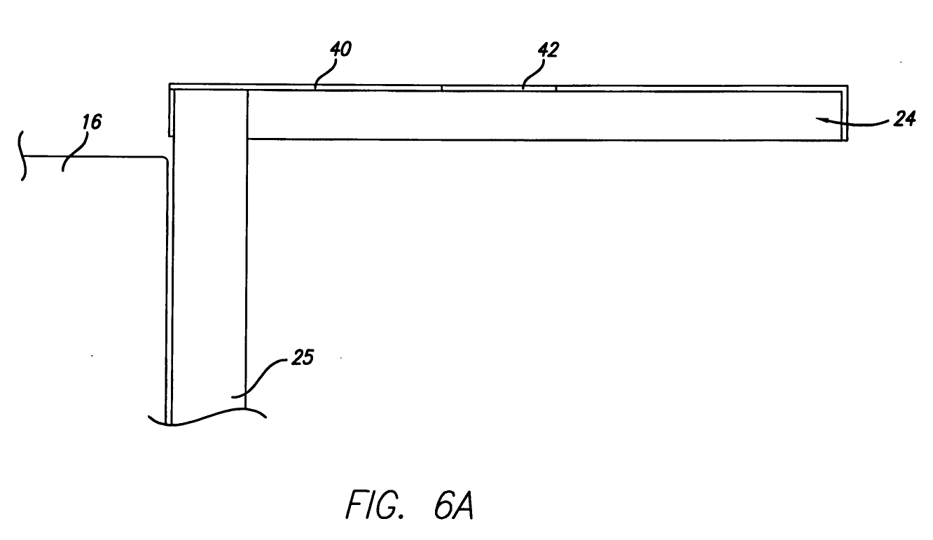

As illustrated in FIGS. 5-6, the platform 24 has a channel 34 formed therein so as to allow the cables 36 from the processor to pass therethrough. In an alternative embodiment, the platform 24 is formed as an entirely hollow structural member. In a further alternative embodiment, as shown in cross-section in FIG. 6A, the platform 24 is formed by milling a solid piece of material leaving only a top surface 40, having an aperture 42 formed therethrough, and sidewalls of the solid piece. The top surface 40 of the platform 24 has an aperture 42 formed therein through which the cables 36 pass from the platform 24 to the first arm 26, as shown in FIGS. 4-6A. The aperture 42 receives a connecting member configured to allow the first arm 26 to be rotatable relative to the platform 24. The aperture 42 is offset from the center of the platform 42 such that the remaining components form a compact mechanism when retracted. The aperture 42 can be located on the platform 24 at any location sufficient to provide a pathway for the cables connected to the control panel 20 and provide a connection sufficient to withstand the stresses associated with the movement of the remaining components of the ergonomic positioning mechanism 22.

A first v-ring 44 allows for rotation of members connected thereto and is disposed within the aperture 42 in the top surface 40 of the platform 24, as illustrated in FIGS. 4-6. A plurality of bearings 46 are disposed about the edge of the v-ring 44 such that the bearings 46 of the platform 24 receive an edge of the v-ring 44, thereby limiting the first arm 26 to rotational movement relative to the platform 24. The first v-ring 44 provides a rotatable connection between the platform 24 and the first arm 26. Other rotatable connecting mechanisms can be used to operatively connect the first arm 26 to the platform 24, thereby allowing the first arm 26 to rotate relative to the platform 24. A first pin 48 is disposed on the top surface 40 of the platform 24, as illustrated in FIG. 5. The first pin 48 extends outward from the top surface 40 and is shaped and sized to be received by the first arm 26 in order to limit the rotational movement of the first arm 26 relative to the platform 24.

The first arm 26 is operatively connected to the platform 24 and forms a first link between the platform 24 and the control panel 20, as shown in FIG. 4. The first v-ring 44 provides a first rotational axis 50 for the ergonomic positioning mechanism 22 between the first arm 26 and the platform 24, as shown in FIGS. 4-5.

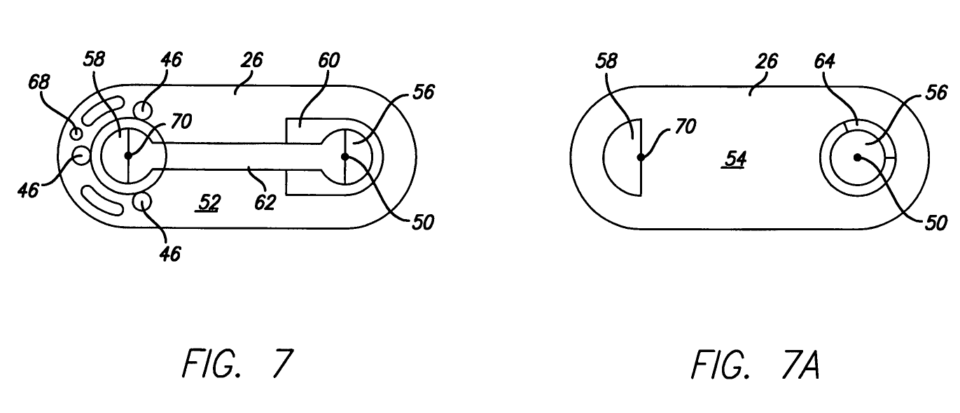

In one embodiment, the first arm 26 is a solid elongated link having opposing distal ends in which the distal ends of the first arm 26 are rounded, as illustrated in FIGS. 7-7A. The first arm 26 has a top surface 52 and a bottom surface 54 and is formed of a solid piece of metal having a first aperture 56 and a second aperture 58, wherein the apertures 56, 58 are formed through the thickness of the first arm 26. The first and second apertures 56, 58 are generally aligned along the longitudinal centerline of the first arm 26 and disposed adjacent to the opposing distal ends of the first arm 26. The top surface 52 of the first arm 26 has a lowered portion 60 formed around the first aperture 56. A channel 62 extends from the top surface 52 toward the center of the first arm 26 and extends between the first aperture 56 and the second aperture 58. The channel 62 can be formed through the entire thickness of the first arm 26, through a portion of the thickness of the first arm 26, or have a stepped thickness such that the channel 62 extends from the top surface 52 of the first arm 26 a different distance at different locations between the first aperture 56 and the second aperture 58. The channel 62 is formed in the first arm 26 and allows the cables to be received in the first aperture 56, extend through the channel 62, and exit the first arm 26 through the second aperture 58. A cover (not shown) is disposed in the lowered portion 60 surrounding the first aperture 56 and in the channel 62 such that the cover is substantially co-planar with the top surface 52 of the first arm 26.

The first aperture 56 of the first arm 26 is aligned with the aperture 42 of the platform 24 such that the first rotational axis 50 passes through both apertures 42, 56, as illustrated in FIG. 4. The bottom surface 54 of the first arm 26 is directed toward, and adjacent to, the top surface 40 of the platform 24. The first v-ring 44 disposed in the aperture 42 of the platform 24 is received by the first aperture 56 of the first arm 26, thereby allowing the first arm 26 to rotate about the first rotational axis 50 relative to the platform 24. The first arm 26 is operatively connected to the platform 24 in a cantilevered manner by way of the first v-ring 44 and bearings 46. The structure of the first arm 26 for connecting the first arm 26 to the platform 24 is different if an alternative rotating connecting mechanism is used to connect the first arm 26 to the platform 24. A second v-ring 66 is disposed in the second aperture 58 in the first arm 26 to allow the second arm 28 to rotate relative to the first arm 26.

The bottom surface 54 of the first arm 26 has a groove 64 formed therein, as shown in FIG. 7A. The groove 64 receives the first pin 48 that extends from the top surface 40 of the platform 24. The groove 64 has opposing end walls that limit the rotation of the first arm 26 relative to the platform 24 by way of abutting contact between the first pin 48 and the end walls of the groove 64. The groove 64 is shaped as an arc, and is located adjacent to the first aperture 56 of the first arm 26 and substantially the same distance from the first rotational axis 50 as the first pin 48. A second pin 68 is located on the top surface 52 of the first arm 26 and extends upward therefrom, as shown in FIG. 7.

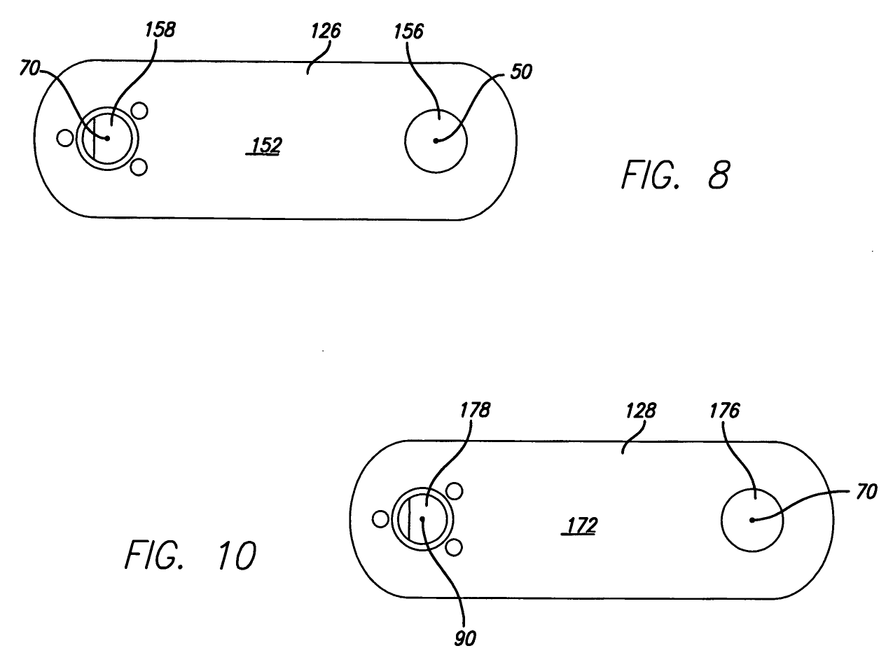

In an alternative embodiment, illustrated in FIG. 8, the first arm 126 is an elongated member having a top surface 152, bottom surface (not shown), a first aperture 156, and a second aperture 158. Both the first and second apertures 156, 158 of the first arm 126 are formed through the entire thickness of the first arm 126 and are generally aligned along the longitudinal length of the first arm 126. The first aperture 156 of the first arm 126 receives the first v-ring 44, thereby rotatably connecting the first arm 126 to the platform 24. The second v-ring 66 is disposed in the second aperture 158 of the first arm 126 to rotatably connect the first arm 126 to the second arm 28. In another alternative embodiment, the first aperture 156 of the first arm 126 is formed through only the bottom surface of the first arm 126 and the second aperture 158 of the first arm 126 is formed through only the top surface 152 of the first arm 126.

The second arm 28 is operatively connected to the first arm 26 and forms a second link between the platform 24 and the control panel 20, as shown in FIG. 4. In one embodiment, the second arm 28 is operatively attached to the first arm 26 in a rotatable manner by way of the second v-ring 66 connector. The second v-ring 66 provides a second rotational axis 70 for the ergonomic positioning mechanism 22 between the first arm 26 and the second arm 28, as shown in FIGS. 4, 7-7A, and 9-9A.

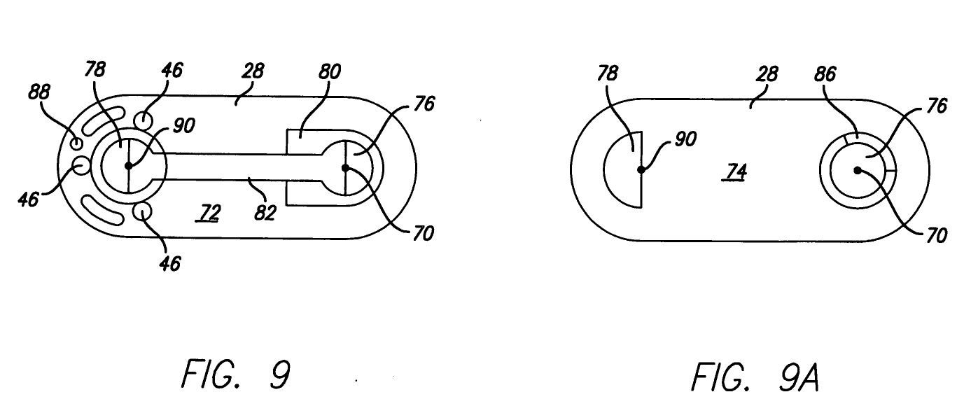

In one embodiment, the second arm 28 is a solid elongated link having opposing distal ends in which the distal ends of the second arm 28 are rounded, as illustrated in FIGS. 9-9A. The second arm 28 has a top surface 72 and a bottom surface 74 and is formed of a solid piece of metal having a first aperture 76 and a second aperture 78, wherein the apertures 76, 78 are formed through the thickness of the second arm 28. The first and second apertures 76, 78 are generally aligned along the longitudinal centerline of the second arm 28 and disposed adjacent to the opposing distal ends of the second arm 28. The top surface 72 of the second arm 28 has a lowered portion 80 formed around the first aperture 76. A channel 82 extends from the top surface 72 of the second arm 28 toward the center of the second arm 28 and extends between the first aperture 76 and the second aperture 78. The channel 82 can be formed through the entire thickness of the second arm 28, through a portion of the thickness of the second arm 28, or have a stepped thickness such that the channel 82 extends from the top surface 72 of the second arm 28 a different distance at different locations between the first and second apertures 76, 78 of the second arm 28. The channel 82 is formed in the second arm 28 and allows the cables to be received in the first aperture 76, extend through the channel 82, and exit the second arm 28 through the second aperture 78. A cover (not shown) is disposed in the lowered portion 80 of the top surface 72 of the second arm 28 surrounding the first aperture 76 and in the channel 82 such that the cover is substantially co-planar with the top surface 72 of the second arm 28.

The first aperture 76 of the first arm 28 is aligned with the second aperture 58 of the first arm 26 such that the second rotational axis 70 passes through both apertures 58, 76, as illustrated in FIGS. 4, 7-7A, and 9-9A. The bottom surface 74 of the second arm 28 is directed toward, and adjacent to, the top surface 52 of the first arm 26. The second v-ring 66 disposed in the second aperture 58 of the first arm 26 is received by the first aperture 76 of the second arm 28, thereby allowing the second arm 28 to rotate about the second rotational axis 70 relative to the first arm 26. The second arm 28 is operatively connected to the first arm 26 in a cantilevered manner by way of the second v-ring 66 and bearings 46. The structure of the second arm 28 for connecting the second arm 28 to the first arm 26 is different if an alternative rotating connecting mechanism is used to connect the second arm 28 to the first arm 26. A third v-ring 84 is disposed in the second aperture 78 in the second arm 28 to allow the table 32 to rotate relative to the second arm 28.

The bottom surface 74 of the second arm 28 has a groove 86 formed therein, as shown in FIG. 9A. The groove 86 receives the second pin 68 that extends from the top surface 52 of the first arm 26. The groove 86 in the second arm 28 has opposing end walls that limit the rotation of the second arm 28 relative to the first arm 26 by way of abutting contact between the second pin 68 and the end walls of the groove 86 in the second arm 28. The groove 86 is shaped as an arc, and is located adjacent to the first aperture 76 of the second arm 28 and substantially the same distance from the second rotational axis 70 as the second pin 68. A third pin 88 is located on the top surface 72 of the second arm 28 and extends upward therefrom, as shown in FIG. 9.

In an alternative embodiment, illustrated in FIG. 10, the second arm 128 is an elongated member having a top surface 172, bottom surface (not shown), a first aperture 176, and a second aperture 178. Both the first and second apertures 176, 178 of the second arm 128 are formed through the entire thickness of the second arm 128 and are generally aligned along the longitudinal length of the second arm 128. The first aperture 176 of the second arm 128 receives the second v-ring 66, thereby rotatably connecting the second arm 128 to the first arm 26. The third v-ring 84 is disposed in the second aperture 178 of the second arm 128 to rotatably connect the table 32 to the second arm 28. In another alternative embodiment, the first aperture 176 of the second arm 128 is formed through only the bottom surface of the second arm 128 and the second aperture 178 of the second arm 128 is formed through only the top surface 172 of the second arm 128.

The table 32 is operatively connected to the second arm 28, as illustrated in FIG. 4, and the table 32 is configured to allow the control panel 20 to be disposed thereon. In one embodiment, the control panel 20 is integrally formed with the table 32, thereby forming a single mechanism that allows a user to input data to the ultrasound system. In an alternative embodiment, the control panel 20 is disposed on top of the table 32 such that the control panel 20 can be relocated on the table 32 or removed therefrom. The table 32 is attached to the second arm 28 in an independently rotatable manner by way of the third v-ring 84 connector. The table 32 is operatively connected to the second arm 28 in a cantilevered manner by way of the third v-ring 84 and bearings 46. Any other type of rotatable connection sufficient to withstand the load resulting from the table 32 being connected to the second arm 28 in a cantilevered manner can be used. The third v-ring 84 provides a third rotational axis 90 for the ergonomic positioning mechanism 22 between the table 32 and the second arm 28, as illustrated in FIGS. 4 and 11.

In one embodiment, the table 32 has a bucket-type shape in which the rear edge of the table 32 extends upward a greater distance than the from edge of the table 32, thereby providing sloped opposing side edges, as shown in FIG. 4, and the control panel 20 is integrally formed with the table 32. In an alternative embodiment, the table 32 is made as an aluminum casting having a hollow center wherein a separate control panel 20 can be disposed on the top surface of the table 32. The table 32 has a bottom surface 92, as shown in FIGS. 4 and 11. A pair of handles 94 are disposed adjacent the forward edge of the table 32. The handles 94 allow a user to grasp the table 32 so as to move the table 32 to a comfortable position. The bottom surface 92 of the table 32 is directed toward, and adjacent to, the top surface 72 of the second arm 28, as illustrated in FIG. 4. The aperture 96 of the table 32 is formed through the bottom surface 92 of the table 32 and receives the third v-ring 84 and rotatably connects the table 32 to the second arm 28. The cables 36 exit from the second aperture 78 of the second arm 28, pass through the third v-ring 84, and pass through the aperture 96 of the table 32 to be connected to the control panel 20. By allowing the cables to pass through the interior of each of the components of the ergonomic positioning mechanism 22, the cables 36 are hidden, thereby preventing damage to the cables and providing a more aesthetic appearance without having the cables connecting the processor to the control panel 20 and the ultrasound transducer be exposed.

The aperture 96 of the table 32 is located near the center of gravity of the table 32 in order to maintain the table 32 and control panel 20 in a substantially balanced condition, as illustrated in FIG. 11. The aperture 96 of the table 32 is aligned with the second aperture 78 of the second arm 28, and the third rotational axis 90 of the ergonomic positioning mechanism 22 passes through these corresponding apertures 78, 96, as shown in FIGS. 4, 9-9A, and 11. The structure of the table 32 for connecting the table 32 to the second arm 28 is different if an alternative connecting mechanism is used to connect the table 32 to the second arm 28.

The third pin 88 is disposed on the top surface 72 of the second arm 28, as illustrated in FIG. 9. The third pin 88 extends outward from the top surface 72 of the second arm 28 and is operably received by the table 32 in order to limit the rotation of the table 32 relative to the second arm 28. The third pin 88 is located adjacent to the second aperture 78 of the second arm 28. A slot 98 is formed in the bottom surface 92 of the table 32, as shown in FIG. 11, and the slot 98 receives the third pin 88 extending from the second arm 28. The slot 98 provides a limit to the rotational movement of the table 32 relative to the second arm 28.

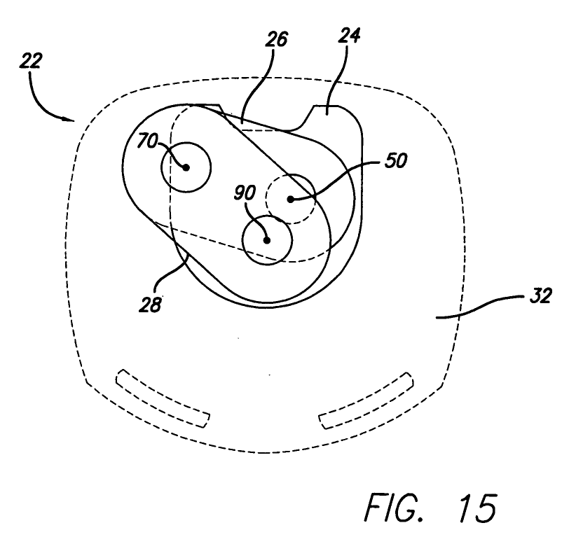

In one embodiment, a stop mechanism 30 is attached to the platform 24, as illustrated in FIG. 12, and is configured to secure the ergonomic positioning mechanism 22 in a home position, or a first operating position, as illustrated in FIGS. 13-15. One embodiment of the stop mechanism 30 includes a mounting member 100, plunger 102, and a cam 104. The mounting member 100 is connected to the platform 24, but the mounting member 100 can alternatively be connected to the housing 26 of the ultrasound component cart 10 when the lift mechanism 25 is not present. As illustrated in FIG. 11, the bottom surface 92 of the table 32 includes a detent 106 formed therein, wherein the detent 106 receives the plunger 102 that extends from the stop mechanism 30 when the ergonomic positioning mechanism 22 is located in the home position, as illustrated in FIG. 15. In one embodiment, the detent 106 is frustoconically-shaped and the plunger 104 is shaped in a corresponding manner. The detent 106 and the plunger 104 can have any other shape sufficient to secure the table 32 in the home position. The plunger 102 is spring-loaded such that it is actuatable downward toward the mounting member 100.

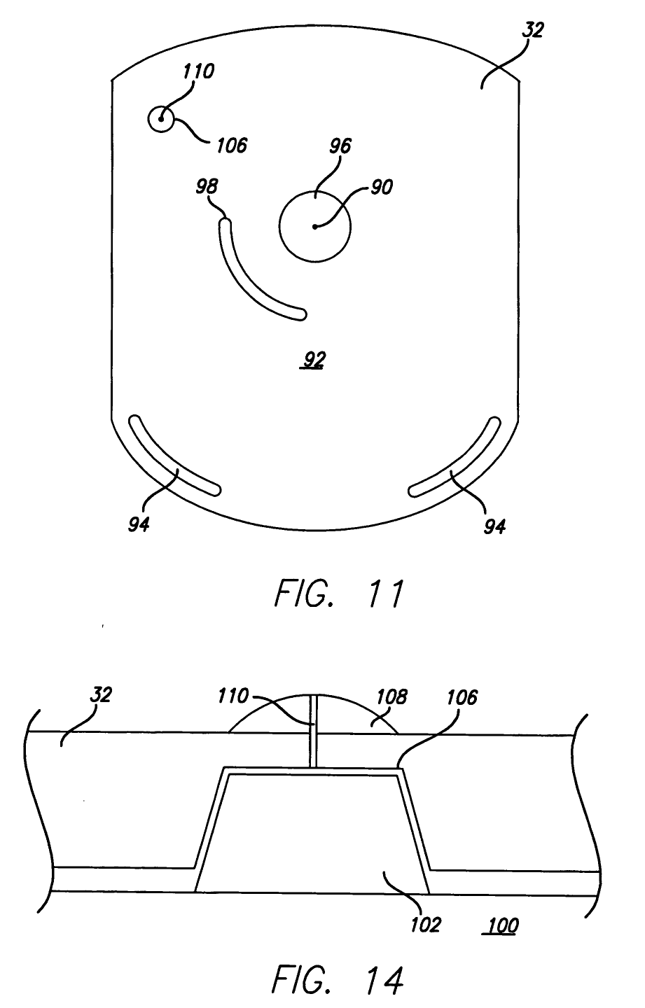

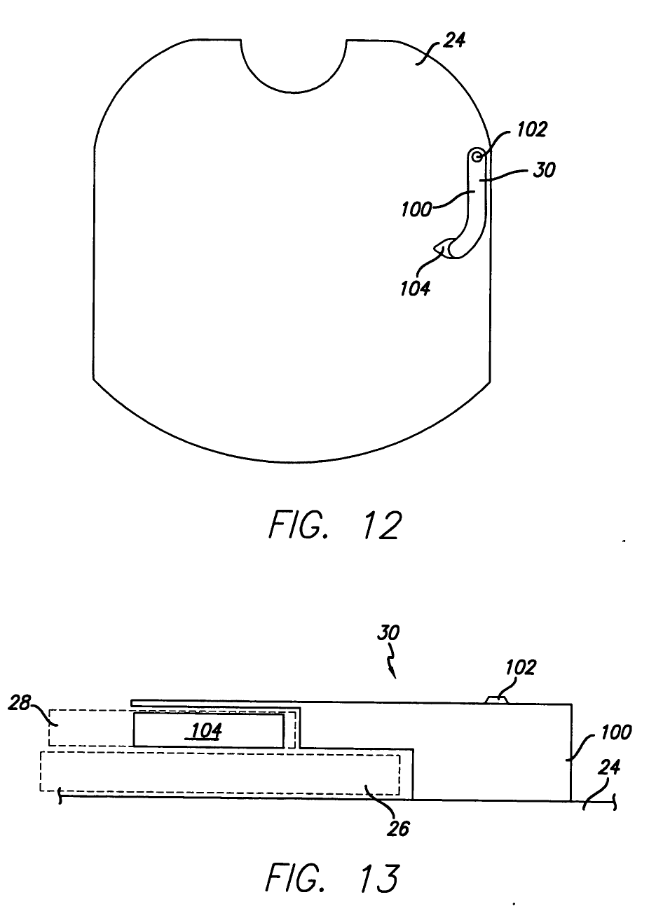

FIG. 15 illustrates the ergonomic positioning mechanism 22 located in the home position, whereby the table 32 is located in a secured position such that the table 32 remains substantially stationary relative to the platform 24. When in the home position, the ultrasound component cart 10 can be maneuvered from location to location without movement of the components of the ergonomic positioning mechanism 22. To release the ergonomic positioning mechanism 22 from the home position, the user depresses a button 108 located on the table 32, as shown in FIG. 14. In one embodiment, the button 108 is connected to a shaft 110 that extends through the thickness of the table 32 into the detent 106 such that when the button 108 is pressed, the shaft 110 contacts the spring-loaded plunger 102 thereby actuating the plunger 102 so as to disengage the plunger 102 from the detent 106 and allowing the table 32 to be moved relative to the platform 24. The shaft 110 has a length sufficient to ensure that the plunger 102 is completely disengaged from the detent 106, thereby allowing the table 32 to be moveable.

In an alternative embodiment, the table 32 can be released from the home position by way of a trigger (not shown) located adjacent to a handle 94 in the table 32. The trigger is attached to a substantially rigid wire that actuates a pusher that engages the plunger 102 so as to depress the plunger 102, thereby releasing the table 32. In a further alternative embodiment, a trigger is attached to a pusher by way of a flexible wire such that actuation of the trigger causes the pusher to engage the plunger 102 to depress the plunger 102 and release the table 32. In yet another alternative embodiment, a button (not shown) can be located on the table 32, wherein the button is connected to a solenoid that is actuated to depress the plunger 102 to release the table 32.

FIGS. 15-20 show a top view of the ergonomic positioning mechanism 22 in various orientations, allowing a user to maneuver the table 32 to an ergonomic, comfortable position. The rotational movement of the various components of the ergonomic positioning mechanism allows the table to be positioned in a multitude of different orientations not shown in the figures. The different orientations may be limited by the range of motion allowed by each of the components. The ergonomic positioning mechanism 22 may provide the user with the ability to position the ultrasound component cart 10 within a crowded room yet may provide the user with access to the control panel 20 to input data to the processor while simultaneously maneuvering the ultrasound transducer without the need for contorting the user's body for simultaneous access to both. When the ultrasound component cart 10 is secured at a desired location, the ergonomic positioning mechanism 22 allows the table 32 to translate relative to the component cart in a planar manner such that the table 32 appears to have a floating movement. The ergonomic positioning mechanism 22 allows the table 32 to be rotatable relative to the component cart 10 when the table 32 is located at any position other than the home position. Vertical adjustment of the table 32 relative to the component cart 10 can also be achieved with additional components added to those of the ergonomic positioning mechanism 22 described above.

In one embodiment, the table 32 is rotatable relative to the platform 24 between about one hundred degrees clockwise and counter-clockwise with respect to the position of the table 32 in the home position, as shown in FIGS. 19-21. In other words, the table 32 is rotatable to at least a perpendicular orientation relative to the home position, as shown in FIG. 21 such that the user has access to the control panel 20 while simultaneously performing an ultrasound examination. In one embodiment, the monitor 18 is attached to the table 32 in a fixed manner. In an alternative embodiment, the monitor 18 is attached to an adjustment mechanism that allows the monitor 18 to be adjusted relative to the table 32. The table 32 is rotatable to at least a perpendicular orientation in the opposite direction with respect to the home position, thereby allowing the user to be positioned adjacent to the opposing side of the ultrasound component cart 10.

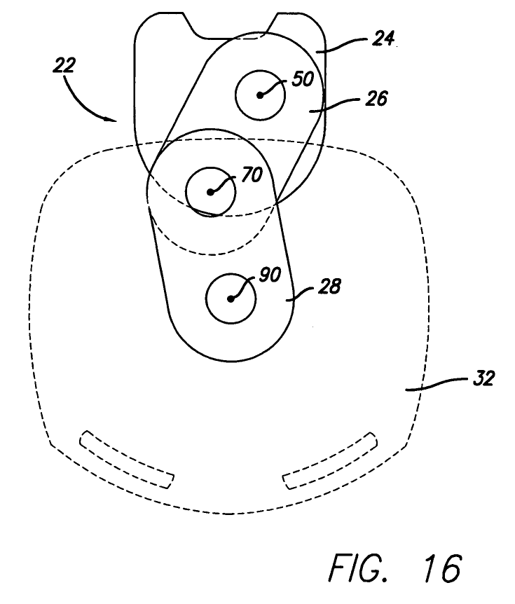

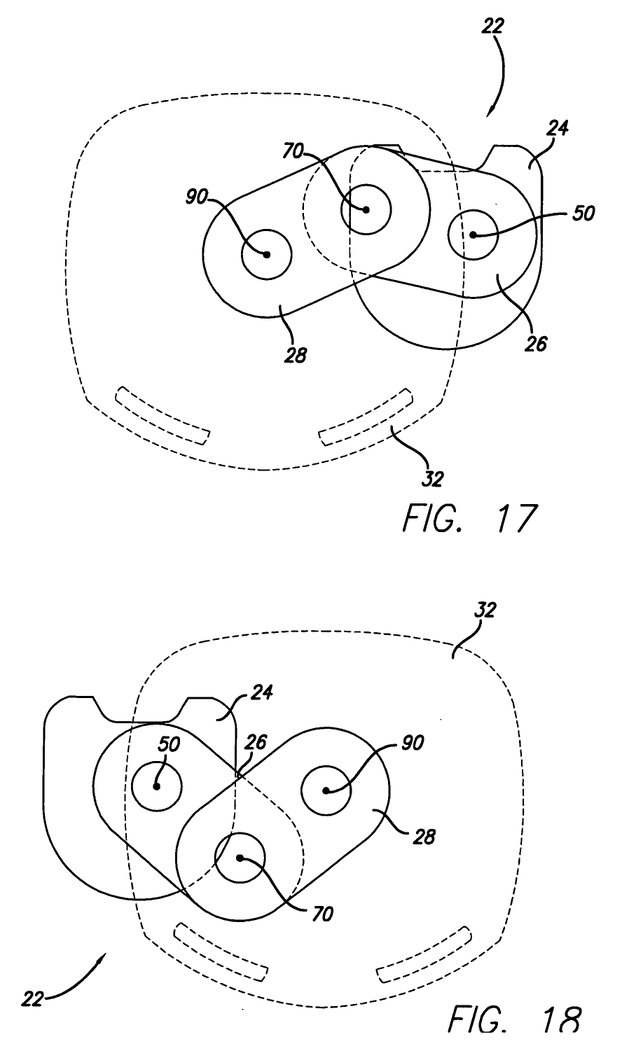

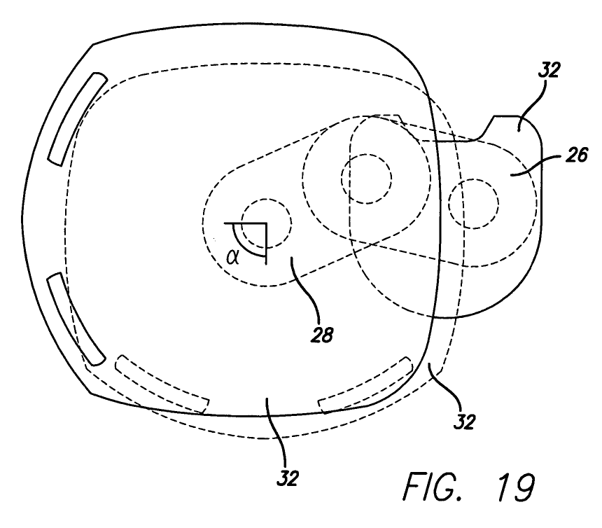

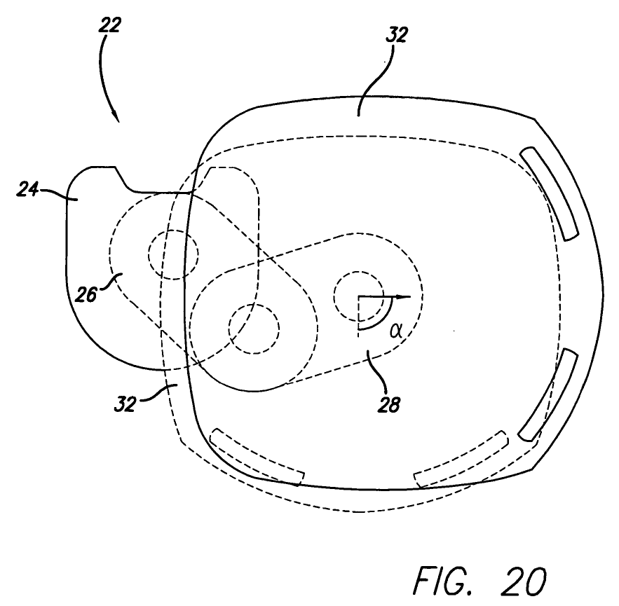

In the home position, as shown in FIG. 15, the ergonomic positioning mechanism 22 is secured such that the table 32 is prevented from any movement as a result of the plunger 102 of the stop mechanism 30 being disposed within the detent 106 of the table 32 (FIG. 14). Once the ergonomic positioning mechanism 22 is released from the home position, the table 32 is translatable in the fore/aft direction relative to the platform 24, as illustrated in FIG. 16. The table 32 is also translatable in the lateral directions relative to the platform 24, as illustrated in FIGS. 17-18. Furthermore, the table 32 is rotatable relative to the second arm 28, as illustrated in FIGS. 19-20.

FIG. 16 illustrates the alignment of the first and second arms 26, 28 when the table 32 has been actuated to the forwardmost location relative to the platform. In one embodiment, the table 32 is configured to travel about twelve (12) inches in the forward direction. The length of both the first and second arms 26, 28 can be increased or decreased, thereby increasing or decreasing the range of movement of the table 32. Additional linkages, or arms, can be added to the ergonomic positioning mechanism, thereby increasing the fore/aft range of movement of the table 32 relative to the platform 24. Because of the arrangement of the components of the ergonomic positioning mechanism 22, when the table 32 is moved and directed toward the component cart 10 from any actuated position away from the home position, the first arm 26 is rotateable in a clockwise manner such that the table 32 is guided into the home position in a self-guided manner. The self-guided ergonomic positioning mechanism 22 allows the table 32 to be guided back to the home position without the need for the user to be able to view the arms 26, 28 in order to align the table 32 in the home position. The table 32 is self-guided to the home position by simply pushing the table toward the vicinity of the home position, and as the arms 26, 28 are directed to the home position, so to is the table 32.

FIGS. 17-18 illustrate exemplary locations of the lateral range of movement of the ergonomic positioning mechanism and the alignment of the first and second arms 26, 28 that allow the table 32 to attain those positions. In one embodiment, the table 32 travels about ten and one-half inches (10.5″) in both of the lateral directions relative to the platform 24. The length of both the first and second arms 26, 28 can be increased or decreased, thereby increasing or decreasing the range of movement of the table 32. Additional linkages, or arms, can be added to the ergonomic positioning mechanism 22, thereby increasing the lateral range of movement of the table 32 relative to the platform 24.

FIGS. 19-20 illustrate an exemplary range of rotational movement of the table 32 relative to the platform 24. The angle of rotation a of the table 32 relative to the fore/aft direction of the platform 24 is between about one hundred degrees in the clockwise direction (FIG. 19) and about one hundred degrees in the counter-clockwise direction (FIG. 20). The range of rotational movement of the table 32 is sufficient to allow the table 32 to be rotated to a position such that a user can be in a seated position that is substantially perpendicular to the component cart 10 and have access to the control panel 20 while performing an ultrasound, as illustrated in FIG. 21. Other ranges of movement may be provided.

In one embodiment, the first arm 26 is constrained in the rotational range of movement relative to the platform 24, the second arm 28 is constrained in the rotational movement relative to the first arm 26, and the table 32 is constrained in the rotational range of movement relative to the second arm 28. Any of the members of the ergonomic positioning mechanism 22 can be constrained with respect the relative movement of adjoining members. Any or all of the members of the ergonomic positioning mechanism 22 can have their movement constrained or have complete freedom of movement with respect to adjoining members.

The range of movement of the first arm 26 relative to the platform 24 is constrained by the first pin 48 disposed on the top surface 40 of the platform 24, as shown in FIGS. 5 and 7A. The first pin 48 is received in groove 64 (FIG. 7A) formed in the bottom surface 54 of the first arm 26. The ends of the groove 64 of the first arm 26 determine the amount of rotational movement of the first arm 26 relative to the platform 24. Any other type of mechanism configured to limit the rotational movement of the first arm 26 relative to the platform 24 can be used.

The range of movement of the second arm 28 relative to the first arm 26 is constrained by the second pin 68 disposed on the top surface 52 of the first arm 26, as shown in FIGS. 7 and 9A. The second pin 68 is received in groove 86 (FIG. 9A) formed in the bottom surface 74 of the second arm 28. The ends of the groove 86 of the second arm 28 determine the amount of rotational movement of the second arm 28 relative to the first arm 26. Any other type of mechanism configured to limit the rotational movement of the second arm 28 relative to the first arm 26 can be used.

The range of movement of the table 32 relative to the second arm 28 is constrained by the third pin 88 disposed on the top surface 72 of the second arm 28, as shown in FIGS. 9 and 11. The third pin 88 is received in a slot 98 (FIG. 11) formed in the bottom surface 92 of the table 32. The ends of the slot 98 determine the amount of rotational movement of the table 32 relative to the second arm 28. Any other type of mechanism configured to limit the rotational movement of the table 32 relative to the second arm 28 can be used.

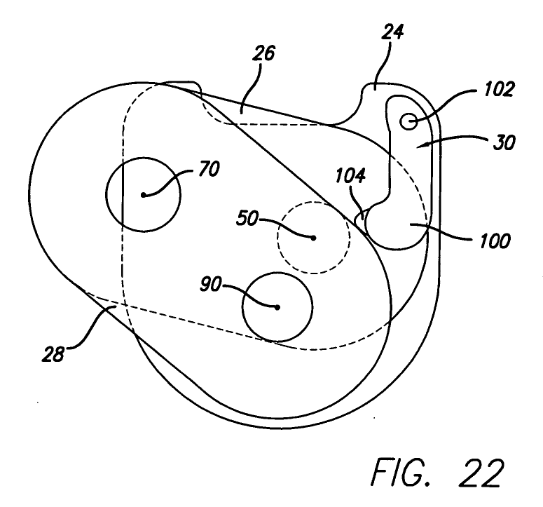

FIG. 22 illustrates the first arm 26 and the second arm 28 located in the home position. When in the home position, the ergonomic positioning mechanism is a four-bar mechanism wherein the first linkage is the first arm 26, the second linkage is the second arm 28, the third linkage is the table 32 (not shown), and the fourth bar is the combination of the platform 24 and the stop mechanism 30. The four-bar linkage has four points of rotation: the first rotational axis 50, the second rotational axis 70, the third rotational axis 90, and the plunger 102-detent 106 connection. Although the table 32 is positioned such that there is no translational movement relative to the platform 24, there may still be some slight movement of the first arm 26 and the second arm 28 as well as rotation of the table about the two points of rotation thereon. The cam 104 located on the stop mechanism 30, as shown in FIGS. 13 and 22, eliminates or reduces the extraneous movement of the components of the ergonomic positioning mechanism 22 when located in the home position. The cam 104 extends from the mounting member 100 and contacts a side edge of the second arm 28. By preventing or limiting any movement of the components of the ergonomic positioning mechanism 22 when located in the home position, the entire ultrasound system is more secure, thereby allowing the ultrasound system to be transported from location to location safely. Any other type of mechanism configured to secure the components of the ergonomic positioning mechanism in the home position and reduce any movement of the components while in the home position can be used.

While preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

1. An ergonomic positioning mechanism comprising:
a platform operatively connected to a component cart, wherein electronic components of an ultrasound system are located in said component cart;
a first arm operatively connected to said platform;
a second arm operatively connected to said first arm; and
a table operatively connected to said second arm and said table is moveable relative to said platform.

2. The ergonomic positioning mechanism of claim 1, wherein said first arm is connected to said platform by way of a rotatable mechanism.

3. The ergonomic positioning mechanism of claim 2, wherein said rotatable mechanism connecting said first arm to said platform is a v-ring.

4. The ergonomic positioning mechanism of claim 1, wherein said second arm is connected to said first arm by way of a rotatable mechanism.

5. The ergonomic positioning mechanism of claim 4, wherein said rotatable mechanism connecting said second arm to said first arm is a v-ring.

6. The ergonomic positioning mechanism of claim 1, wherein said table is connected to said second arm by way of a rotatable mechanism.

7. The ergonomic positioning mechanism of claim 6, wherein said rotatable mechanism connecting said table to said second arm is a v-ring.

8. The ergonomic positioning mechanism of claim 1 further including a stop mechanism.

9. The ergonomic positioning mechanism of claim 1, wherein said table is translatable relative to said platform in a planar manner.

10. The ergonomic positioning mechanism of claim 9, wherein said table is translatable in a lateral direction to about ten and one-half inches relative to said platform.

11. The ergonomic positioning mechanism of claim 9, wherein said table is translatable in the forward direction to about twelve inches relative to the platform.

12. The ergonomic positioning mechanism of claim 9, wherein said table is rotatable relative to said platform.

13. The ergonomic positioning mechanism of claim 12, wherein said table is rotatable to about one hundred degrees in the clockwise direction relative to said platform.

14. The ergonomic positioning mechanism of claim 12, wherein said table is rotatable to about one hundred degrees in the counter-clockwise direction relative to said platform.

15. A diagnostic ultrasound system comprising:
a component cart in which said diagnostic ultrasound system is located; and
an ergonomic positioning mechanism connected to said component cart, said ergonomic positioning mechanism including a table configured to support a control panel connected to said diagnostic ultrasound system by at least one cable, wherein said ergonomic positioning mechanism includes a stop mechanism operable to maintain said table in a substantially fixed position during transport of said ultrasound system.

16. The diagnostic ultrasound system of claim 15, wherein said table is releasable from said substantially fixed position.

17. The diagnostic ultrasound system of claim 16, wherein said stop mechanism includes a plunger operable to engage a corresponding detent in said table when said table is in said substantially fixed position.

18. The diagnostic ultrasound system of claim 17, wherein a button disposed on said table releases said table from said substantially fixed position.

19. The diagnostic ultrasound system of claim 15, wherein said ergonomic positioning mechanism is configured to conceal said at least one cable connecting said control panel to said ultrasound system.

20. An ergonomic positioning mechanism comprising:
a platform attachable to an ultrasound component cart;
a first arm rotatably connected to said platform;
a second arm rotatably connected to said first arm;
a table rotatably connected to said second arm, said table is translatable and rotatable relative to said platform; and
a stop mechanism configured to releasably engage said table in a first operative position.

21. An ergonomic positioning mechanism comprising:
a control panel rotatably connected to a pair of arms, wherein said pair of arms are connected in series, said pair of arms are rotatably attached to a housing.
